SEQUENCE LISTING (6 sequences)

SEQ ID NO: 1 - DNA, 3026 bp, Homo sapiens
tgttgccaccggatgaaaggtggaggaggcagaaacctaaacaagtgaattcaattacatctgaaaaagattaggctaaattaggattcattccttgggccagttccaggaaatatttgtgaaaagttggttgaaagaatactaccatctaaaccctaattttgctattacgtaaaagccccgattcctttgcagtaaaacattagaaaatgttttcctcattttgtttttgctactggtgtgtgacctcttaatttaattttggtttttgctcatataaatcaatggccgtggaaactttttcctgtggtgcagcaaagacaggggaaaagtagaacatcagtaattatattttaggcacttaatttatttaatgagcagttatcaagcacccttaatggttccagccataaactataggctcctcccgctccctcggcctccaggttcaataaggattacatcatggagcaagtatgcggggcagcctgagaatcaaagccgtcagcaccggcggctggttaactgtactgttgctgaaacacatccttcccaccccctccccgcgcccctctccctgccccggccctctgcttttcgtaccattaaggatagcattattggggagaggaaaatctgatcaaaactgtcgaccgccaacgacagattttctatggattctataaaggccaccttaacgatttccctaattaatggatgacatctttggctgaaggagaaacgatggattcgactctgtcttcgtctttgttaaccttctaattaactttcgtaggtcacccaggtctcccaaacctgaaaagtgaagaggcccattgtgcgaggagcagtaaacttactttttctgctggggtacttaaaagggagagcggctataggccgagaaaaataactccatttaaaagctttgtggctgcagagaatacataggtccctcccttagaaaataattgaaacaccttttataccaatatttctatgcataataaaaacaaattagcaaaatgcatctaaattcagcagcactttcaaattaccttttttggaatgtaagcctctgcaagtcaacaaaaggagttgggtttagagagaaaattgcaactgcaacagctatcagcctttatgctgagagagcaagttacacctgaaattttagaggctaaggagaaatgtctgcaacctacctcctcagttgtgaactcctgtacctgaaaggccaaatcctcctcctcccagcaggcaatcatcttatgccctgaagcatgagatctgattacccctatcatcatcttagcttgcatagatacagatgttattaacggctgtagtttatccaacccttttttaaaacgcagctgcagcatttgacttggggacctcctgtgaatcccactggttgactccgtcttgcacaaagcacgtggggtttgggggagggagaagagagagggaggtttgttttgttttggagggttttttttttttttttttgaatcagagttccctaaatagccactcggcatctttgtaagtgatgtgaattctgaaattacttactagaataggacaacagagtactgtgtgaactagatctgttggaaacaaagcatagtagaatgtatgatgcttttcacttcattggcagagatcaaatatgtggtcctgatatactcatccacataataaagaattgtagcttattctgaaatatattggccacattcaaactggagtacctggttttacataacaattgtttctacagaagaccccatcctcagccctgactttccgtagatatgtgcaaaatgagtaaattactcacctcgggccagatccaagttttacccaacagaaggggcaccggaccaagaatgaaccaactcacatggccatgtccggcgcgcacaatcacacgccagcacacagccacccaatttcttccgcgaatctatctggcactctggagagagggggaaaagcgttttgagaaagccccgtcacccctccccttccttcttgccgtgaaatatacgaattcatttttattacgagccgcaccgtcctcaccatcacgcacgcacagagccacactcccatattcacactttctaactcgtaagctccgacagcgcctgcattttctttgggagccgcttggaggttcattaatatcattagcatttaaccccctccctcttcccatcccctccccgcacatggctgacgtcagaccccgccaggagttgggggaaaagctaagtgggccagggacgccctattcccctccccgcggctgcctgtcagagcgcttctggagatattacaggggacccagcccgcagcgacaggcacaaagtcacggggtaatgaacttcggggacccttcgccgctgcgtgcgcggctctccccggaaacccggacctggccgcctcttccctcggaagatttcccagcaatctagttttcccactctgcgcttgggttccggcagcgcggagcccgtctgcctctgagactgcggtagtgttttccttctttccttgggagaccagcggtcggcagagattgcccacactctgcatgcctatgtagagggagagatcgaagactgagtgacaggaatggggaaaaagagggatttcgctccgtaggaaggccattttcgtgtctccatctctgtctttcaacatccctctcttgctgttcttccttcttcctcagtcttcctgtccatctctccatctgtctgtccatgtgtgtgtccatatcaagcagcattcccagcagctgcggttttgcaagagccgggaagaaacttaaggatgcttaaatttccactgttggacgaattctgagcgcccagggagcagcgcagcgcgcgactgacacccacctgtcccgcccaggagccttgcaggctggagggcagctggagagcggcggcgcccggcggcgaggcgggcgctgccggccgggactcgggcagcgcccaccaaccgct

SEQ ID NO: 2 - DNA, 1243 bp, Homo sapiens
cagaagaccccatcctcagccctgactttccgtagatatgtgcaaaatgagtaaattactcacctcgggccagatccaagttttacccaacagaaggggcaccggaccaagaatgaaccaactcacatggccatgtccggcgcgcacaatcacacgccagcacacagccacccaatttcttccgcgaatctatctggcactctggagagagggggaaaagcgttttgagaaagccccgtcacccctccccttccttcttgccgtgaaatatacgaattcatttttattacgagccgcaccgtcctcaccatcacgcacgcacagagccacactcccatattcacactttctaactcgtaagctccgacagcgcctgcattttctttgggagccgcttggaggttcattaatatcattagcatttaaccccctccctcttcccatcccctccccgcacatggctgacgtcagaccccgccaggagttgggggaaaagctaagtgggccagggacgccctattcccctccccgcggctgcctgtcagagcgcttctggagatattacaggggacccagcccgcagcgacaggcacaaagtcacggggtaatgaacttcggggacccttcgccgctgcgtgcgcggctctccccggaaacccggacctggccgcctcttccctcggaagatttcccagcaatctagttttcccactctgcgcttgggttccggcagcgcggagcccgtctgcctctgagactgcggtagtgttttccttctttccttgggagaccagcggtcggcagagattgcccacactctgcatgcctatgtagagggagagatcgaagactgagtgacaggaatggggaaaaagagggatttcgctccgtaggaaggccattttcgtgtctccatctctgtctttcaacatccctctcttgctgttcttccttcttcctcagtcttcctgtccatctctccatctgtctgtccatgtgtgtgtccatatcaagcagcattcccagcagctgcggttttgcaagagccgggaagaaacttaaggatgcttaaatttccactgttggacgaattctgagcgcccagggagcagcgcagcgcgcgactgacacccacctgtcccgcccaggagccttgcaggctggagggcagctggagagcggcggcgcccggcggcgaggcgggcgctgccggccgggactcgggcagcgcccaccaaccgct

SEQ ID NO: 3 - DNA, 419 bp, Homo sapiens
tgttgccaccggatgaaaggtggaggaggcagaaacctaaacaagtgaattcaattacatctgaaaaagattaggctaaattaggattcattccttgggccagttccaggaaatatttgtgaaaagttggttgaaagaatactaccatctaaaccctaattttgctattacgtaaaagccccgattcctttgcagtaaaacattagaaaatgttttcctcattttgtttttgctactggtgtgtgacctcttaatttaattttggtttttgctcatataaatcaatggccgtggaaactttttcctgtggtgcagcaaagacaggggaaaagtagaacatcagtaattatattttaggcacttaatttatttaatgagcagttatcaagcacccttaatggttccagccataaactata

SEQ ID NO: 4 - DNA, 738 bp, Homo sapiens
ggctcctcccgctccctcggcctccaggttcaataaggattacatcatggagcaagtatgcggggcagcctgagaatcaaagccgtcagcaccggcggctggttaactgtactgttgctgaaacacatccttcccaccccctccccgcgcccctctccctgccccggccctctgcttttcgtaccattaaggatagcattattggggagaggaaaatctgatcaaaactgtcgaccgccaacgacagattttctatggattctataaaggccaccttaacgatttccctaattaatggatgacatctttggctgaaggagaaacgatggattcgactctgtcttcgtctttgttaaccttctaattaactttcgtaggtcacccaggtctcccaaacctgaaaagtgaagaggcccattgtgcgaggagcagtaaacttactttttctgctggggtacttaaaagggagagcggctataggccgagaaaaataactccatttaaaagctttgtggctgcagagaatacataggtccctcccttagaaaataattgaaacaccttttataccaatatttctatgcataataaaaacaaattagcaaaatgcatctaaattcagcagcactttcaaattaccttttttggaatgtaagcctctgcaagtcaacaaaaggagttgggtttagagagaaaattgcaactgcaacagctatcagcctttatgctgagagagca

SEQ ID NO: 5 - DNA, 626 bp, Homo sapiens
agttacacctgaaattttagaggctaaggagaaatgtctgcaacctacctcctcagttgtgaactcctgtacctgaaaggccaaatcctcctcctcccagcaggcaatcatcttatgccctgaagcatgagatctgattacccctatcatcatcttagcttgcatagatacagatgttattaacggctgtagtttatccaacccttttttaaaacgcagctgcagcatttgacttggggacctcctgtgaatcccactggttgactccgtcttgcacaaagcacgtggggtttgggggagggagaagagagagggaggtttgttttgttttggagggttttttttttttttttttgaatcagagttccctaaatagccactcggcatctttgtaagtgatgtgaattctgaaattacttactagaataggacaacagagtactgtgtgaactagatctgttggaaacaaagcatagtagaatgtatgatgcttttcacttcattggcagagatcaaatatgtggtcctgatatactcatccacataataaagaattgtagcttattctgaaatatattggccacattcaaactggagtacctggttttacataacaattgtttcta

SEQ ID NO: 6 - DNA, 4350 bp, Homo sapiens
gtgtgttgccaccggatgaaaggtggaggaggcagaaacctaaacaagtgaattcaattacatctgaaaaagattaggctaaattaggattcattccttgggccagttccaggaaatatttgtgaaaagttggttgaaagaatactaccatctaaaccctaattttgctattacgtaaaagccccgattcctttgcagtaaaacattagaaaatgttttcctcattttgtttttgctactggtgtgtgacctcttaatttaattttggtttttgctcatataaatcaatggccgtggaaactttttcctgtggtgcagcaaagacaggggaaaagtagaacatcagtaattatattttaggcacttaatttatttaatgagcagttatcaagcacccttaatggttccagccataaactataggatatcatcggccggcgctattaactcccggggcctcctgcggcggcgaggcggtggcacggctcctcccgctccctcggcctccaggttcaataaggattacatcatggagcaagtatgcggggcagcctgagaatcaaagccgtcagcaccggcggctggttaactgtactgttgctgaaacacatccttcccaccccctccccgcgcccctctccctgccccggccctctgcttttcgtaccattaaggatagcattattggggagaggaaaatctgatcaaaactgtcgaccgccaacgacagattttctatggattctataaaggccaccttaacgatttccctaattaatggatgacatctttggctgaaggagaaacgatggattcgactctgtcttcgtctttgttaaccttctaattaactttcgtaggtcacccaggtctcccaaacctgaaaagtgaagaggcccattgtgcgaggagcagtaaacttactttttctgctggggtacttaaaagggagagcggctataggccgagaaaaataactccatttaaaagctttgtggctgcagagaatacataggtccctcccttagaaaataattgaaacaccttttataccaatatttctatgcataataaaaacaaattagcaaaatgcatctaaattcagcagcactttcaaattaccttttttggaatgtaagcctctgcaagtcaacaaaaggagttgggtttagagagaaaattgcaactgcaacagctatcagcctttatgctgagagagcaagggattttcagatcagaaatatttaatgtgtatattacctattcaaaaaataaattaactcttgagcccaggagtttgagaccagcctgggcaacatagcaggaccccgcctctagaaaacaaaacaaacaaaaaaatgagttaaaacgtacatctacatttttaaaagagaatgggttgccattttccaatgtgctctctgcatagctctgtacaattcaagggtggcccagtcttaaaagtgtctttgttcatttccgtatactaaatagctcattttagacttctcgaagtctacctgggttaaaagacccctaaaatggaaggtacacagtcactctctctttgaaatttttaaatgttttataacaaaaacagaataatgcattctgtgatttaattgttcaaaattaaaattcaacaaactgttaattctgtataaataaaacattcacaggcttcatcagcacgtgcatcaggacaaatgctttgggccttggaattgtgaaaatatttcctaacatttgagatgtgccaagaaagagataaagaaatcaaataagatcttgtattatgcaaattcttcttgctcacaggagtctgatttacttgtatcaatcaatgtaagtgattcacacttcatgagctatattaagtgattgaactaaaacctagggccaatattattctctccccacccctcaacccaggctgtctgtataaataggattggaaattactaggttgaagatatgaaattagtgttgtgtcagtggttcaataccagcaaggaagccctatgtctatctagagatattttgtggagcttaattcagaatacatgtaatttacgtgtacatacatattaaagaattttctggatgtggtagaaacctttgaagtctcttctgcatgttaatttctctgctaatccagatgcctctgctctttgtgacattagagaggtcactgagcaccatcccgataaccctccagaaaacaatgacatgccagtgcagaaccttttttttttctactaaccatttagtattcttttttcttttcaagaaaagccagtcaagtcctaggaggtcttatcactaatactaatttctccgatctgatagaaatgttagatttcaaatcactgtttcacctatcccaacagttacacctgaaattttagaggctaaggagaaatgtctgcaacctacctcctcagttgtgaactcctgtacctgaaaggccaaatcctcctcctcccagcaggcaatcatcttatgccctgaagcatgagatctgattacccctatcatcatcttagcttgcatagatacagatgttattaacggctgtagtttatccaacccttttttaaaacgcagctgcagcatttgacttggggacctcctgtgaatcccactggttgactccgtcttgcacaaagcacgtggggtttgggggagggagaagagagagggaggtttgttttgttttggagggttttttttttttttttttgaatcagagttccctaaatagccactcggcatctttgtaagtgatgtgaattctgaaattacttactagaataggacaacagagtactgtgtgaactagatctgttggaaacaaagcatagtagaatgtatgatgcttttcacttcattggcagagatcaaatatgtggtcctgatatactcatccacataataaagaattgtagcttattctgaaatatattggccacattcaaactggagtacctggttttacataacaattgtttctacaagtgttattcgctcagcgtcggatgggccagctctgcttggggaagctggcggcatcctcccctcggctggtgcccaaacccactgcgcgaaggccgaaggaacgcggaacctccagaagaccccatcctcagccctgactttccgtagatatgtgcaaaatgagtaaattactcacctcgggccagatccaagttttacccaacagaaggggcaccggaccaagaatgaaccaactcacatggccatgtccggcgcgcacaatcacacgccagcacacagccacccaatttcttccgcgaatctatctggcactctggagagagggggaaaagcgttttgagaaagccccgtcacccctccccttccttcttgccgtgaaatatacgaattcatttttattacgagccgcaccgtcctcaccatcacgcacgcacagagccacactcccatattcacactttctaactcgtaagctccgacagcgcctgcattttctttgggagccgcttggaggttcattaatatcattagcatttaaccccctccctcttcccatcccctccccgcacatggctgacgtcagaccccgccaggagttgggggaaaagctaagtgggccagggacgccctattcccctccccgcggctgcctgtcagagcgcttctggagatattacaggggacccagcccgcagcgacaggcacaaagtcacggggtaatgaacttcggggacccttcgccgctgcgtgcgcggctctccccggaaacccggacctggccgcctcttccctcggaagatttcccagcaatctagttttcccactctgcgcttgggttccggcagcgcggagcccgtctgcctctgagactgcggtagtgttttccttctttccttgggagaccagcggtcggcagagattgcccacactctgcatgcctatgtagagggagagatcgaagactgagtgacaggaatggggaaaaagagggatttcgctccgtaggaaggccattttcgtgtctccatctctgtctttcaacatccctctcttgctgttcttccttcttcctcagtcttcctgtccatctctccatctgtctgtccatgtgtgtgtccatatcaagcagcattcccagcagctgcggttttgcaagagccgggaagaaacttaaggatgcttaaatttccactgttggacgaattctgagcgcccagggagcagcgcagcgcgcgactgacacccacctgtcccgcccaggagccttgcaggctggagggcagctggagagcggcggcgcccggcggcgaggcgggcgctgccggccgggac

(19) United States
(12) Patent Application Publication
(10) Pub. No.: US 2018/0208942 A1
(43) Pub. Date: Jul. 26, 2018

(54) NR2E1 MINI-PROMOTERS

(71) Applicant: UNIVERSITY OF BRITISH COLUMBIA, Vancover (CA)

(72) Inventors: Elizabeth M. Simpson, Vancouver (CA); Wyeth W. Wasserman, Vancouver (CA); Daniel Goldowitz, Port Moody (CA); Charles de Leeuw, Seattle, WA (US)

(21) Appl. No.: 15/785,807

(22) Filed: Oct. 17, 2017

Related U.S. Application Data
(63) Continuation of application No. 14/319,557, filed on Jun. 30, 2014, now abandoned.
(60) Provisional application No. 61/842,846, filed on Jul. 3, 2013.

(51) Int. Cl. C12N 15/85; A01K 67/027; A61K 48/00

(52) U.S. Cl. CPC C12N 15/85; A01K 2267/03; A01K 2227/105; A01K 2217/206; A01K 2217/072; C12N 15/8509; A01K 67/0275; C12N 2830/008; C12N 2750/14141; A61K 48/0058

(57) ABSTRACT

Isolated polynucleotides comprising a NR2E1 mini-promoters are provided. The mini-promoter may be operably linked to an expressible sequence, e.g. reporter genes, genes encoding a polypeptide of interest, regulatory RNA sequences such as miRNA, siRNA, anti-sense RNA, etc., and the like. In some embodiments a cell comprising a stable integrant of an expression vector is provided, which may be integrated in the genome of the cell. The promoter may also be provided in a vector, for example in combination with an expressible sequence. The polynucleotides find use in a method of expressing a sequence of interest, e.g. for identifying or labeling cells, monitoring or tracking the expression of cells, gene therapy, etc.

### CROSS REFERENCE

This application claims benefit and is a Continuation of application Ser. No. 14/319,557 filed Jun. 30, 2014, which claims benefit of U.S. Provisional Patent Application No. 61/842,846, filed Jul. 3, 2013, which applications are incorporated herein by reference in their entirety.

### FIELD OF THE INVENTION

The invention relates to gene promoters and regulatory elements. More specifically, the invention relates to novel NR2E1 promoter compositions and related methods.

### BACKGROUND

The NR2E1 gene encodes the nuclear receptor subfamily 2, group E, member 1. NR2E1 functions as an orphan nuclear receptor (no known ligand) transcription factor which represses, or activates, expression of downstream genes. This gene shows a highly localized expression pattern. The NR2E1 transcript can be detected as early as E8 in mouse, with a peak of expression at ˜E12.5, the peak of neurogenesis (Monaghan et al. 1995). The expression is localized to the neuroepithelium during development and later restricted to stem cells of the brain in the adult (Shi et al. 2004), as well as in the Müller glia of the retina (Miyawaki et al. 2004; Schmouth et al. 2012). Current data suggests the expression pattern is conserved in mice and humans.

There is a need for characterized human NR2E1 promoters for gene expression, for instance in human gene therapy applications. It is in particular useful to identify small promoter elements that are sufficient to drive expression in certain cell types, for instance Müller glial cells in the retina. Such small promoter elements, or “mini-promoters” are particularly useful in certain applications, for instance they are more amenable to insertion into viral vectors used in gene therapy applications.

### SUMMARY OF THE INVENTION

The present invention provides novel nucleic acid sequence compositions and methods relating to minimal human NR2E1 promoters. The invention is based in part on the surprising discovery that certain minimal NR2E1 promoter elements are capable of expressing an operably linked genetic sequence in specific cell types, for instance in cells of the brain or eye.

In one embodiment of the invention, there is provided an isolated nucleic acid fragment comprising an NR2E1 mini-promoter, wherein the NR2E1 mini-promoter comprises one or more NR2E1 regulatory elements operably linked in a non-native conformation to a NR2E1 basal promoter. The NR2E1 mini-promoter may have a nucleic acid sequence which is substantially similar in sequence and function to SEQ ID NO:1. The NR2E1 basal promoter may have a nucleic acid sequence which is substantially similar in sequence and function to SEQ ID NO: 2. The NR2E1 regulatory element may have a nucleic acid sequence which is substantially similar in sequence and function to SEQ ID NO: 3, 4, and/or 5. The NR2E1 mini-promoters may further be operably linked to an expressible sequence, e.g. reporter genes, genes encoding a polypeptide of interest, regulatory RNA sequences such as miRNA, siRNA, anti-sense RNA, etc., and the like. Reporter gene sequences include, for example luciferase, beta-galactosidase, green fluorescent protein, enhanced green fluorescent protein, and the like as known in the art. The expressible sequence may encode a protein of interest, for example a therapeutic protein, receptor, antibody, growth factor, transcription factor, and the like. The expressible sequence may encode an RNA interference molecule.

In one embodiment, there is provided an expression vector comprising an NR2E1 mini-promoter, wherein the NR2E1 mini-promoter comprises one or more NR2E1 regulatory elements operably linked in a non-native conformation to an NR2E1 basal promoter. The NR2E1 mini-promoter may have a nucleic acid sequence which is substantially similar in sequence and function to SEQ ID NO: 1. The NR2E1 basal promoter may have a nucleic acid sequence which is substantially similar in sequence and function to SEQ ID NO: 2. The one or more NR2E1 regulatory elements may have a nucleic acid sequence which is substantially similar in sequence and function to SEQ ID NO: 3, 4, and/or 5. The NR2E1 mini-promoter may further be operably linked to an expressible sequence, e.g. reporter genes, genes encoding a polypeptide of interest, regulatory RNA sequences such as miRNA, siRNA, anti-sense RNA, etc., and the like. Reporter gene sequences include, for example luciferase, beta-galactosidase, green fluorescent protein, enhanced green fluorescent protein, and the like as known in the art. The expressible sequence may encode a protein of interest, for example a therapeutic protein, receptor, antibody, growth factor, transcription factor, and the like. The expressible sequence may encode an RNA interference molecule. The expression vector may further comprise a genomic targeting sequence. The genomic targeting sequence may be HPRT.

In one embodiment, there is provided a method for expressing a gene, protein, RNA interference molecule or the like in a cell, the method comprising introducing into the cell an expression vector comprising an NR2E1 mini-promoter element, wherein the NR2E1 mini-promoter element comprises one or more NR2E1 regulatory elements operably linked in a non-native conformation to an NR2E1 basal promoter element. Cells of interest include, without limitation, cells of the peripheral or central nervous system and progenitors thereof, e.g. embryonic stem cells, neural stem cells, neurons, glial cells, astrocytes, etc; and/or cells in the eye and progenitors thereof, e.g. retinal cells, Müller glia cells, etc. The NR2E1 mini-promoter may have a nucleic acid sequence which is substantially similar in sequence and function to SEQ ID NO: 1. The NR2E1 basal promoter may have a nucleic acid sequence which is substantially similar in sequence and function to SEQ ID NO: 2. The NR2E1 regulatory element may have a nucleic acid sequence which is substantially similar in sequence and function to SEQ ID NO: 3, 4, and/or 5. The NR2E1 mini-promoter may further be operably linked to an expressible sequence, e.g. reporter genes, genes encoding a polypeptide of interest, regulatory RNA sequences such as miRNA, siRNA, anti-sense RNA, etc., and the like. Reporter gene sequences include, for example luciferase, beta-galactosidase, green fluorescent protein, enhanced green fluorescent protein, and the like as known in the art. The expressible sequence may encode a protein of interest, for example a therapeutic protein, receptor, antibody, growth factor, transcription factor, and the like. The expressible sequence may encode an RNA interference molecule. The expression vector may thus further comprise a genomic targeting sequence. The genomic targeting sequence may be HPRT.

In one embodiment of the invention, there is provided a method for identifying or labeling a cell, the method comprising introducing into the cell an expression vector comprising an NR2E1 mini-promoter element, wherein the NR2E1 mini-promoter element comprises one or more NR2E1 regulatory element operably linked in a non-native conformation to a NR2E1 basal promoter element, and wherein the expressible sequence comprises a reporter gene. The NR2E1 mini-promoter element may have a nucleic acid sequence substantially similar in sequence and function to SEQ ID NO: 1. The NR2E1 basal promoter element may have a nucleic acid sequence substantially similar in sequence and function to SEQ ID NO: 2. The NR2E1 regulatory element may have a nucleic acid sequence substantially similar in sequence and function to SEQ ID NO: 3, 4, and/or 5. In some embodiments, the cell is a peripheral or central nervous system cell or progenitors thereof, including, without limitation, embryonic stem cells, neural stem cells, glial cells, astrocytes, neurons and the like, and/or cells in the eye and progenitors thereof, e.g. retinal cells, Müller glia, etc. Reporter gene sequences include, for example luciferase, beta-galactosidase, green fluorescent protein, enhanced green fluorescent protein, and the like as known in the art. The expressible sequence may encode a protein of interest, for example a therapeutic protein, receptor, antibody, growth factor, transcription factor, RNA interference molecule and the like.

In one embodiment of the invention, there is provided a method for monitoring or tracking the development or maturation of a cell, the method comprising: 1) introducing into the cell an expression vector comprising an NR2E1 mini-promoter element operably linked to an expressible sequence, wherein the NR2E1 mini-promoter element comprises one or more NR2E1 regulatory elements operably linked in a non-native conformation to an NR2E1 basal promoter element, and wherein the expressible sequence comprises a reporter gene; and 2) detecting the expression of the reporter gene in the cell of in progeny of the cell as a means of determining the lineage, identity or developmental state of the cell or cell progeny. The NR2E1 mini-promoter element may have a nucleic acid sequence substantially similar in sequence and function to SEQ ID NO: 1. The NR2E1 basal promoter element may have a nucleic acid sequence substantially similar in sequence and function to SEQ ID NO: 2. The NR2E1 regulatory element may have a nucleic acid sequence substantially similar in sequence and function to SEQ ID NO: 3, 4, and/or 5. In some embodiments, the cell is a peripheral or central nervous system cell or progenitors thereof, including, without limitation, embryonic stem cells, neural stem cells, glial cells, neurons and the like. In some embodiments, the cell is an eye cell or progenitor thereof, including without limitation a retinal cell, a Müller glial cell, and the like.

In certain embodiments of the invention, there is thus provided a method of treatment of a subject having a disease or condition of the eye, the method comprising administering to the subject a therapeutically effective dose of a composition comprising an NR2E1 mini-promoter element, wherein the NR2E1 mini-promoter element comprises one or more NR2E1 regulatory elements operably linked in a non-native conformation to an NR2E1 basal promoter element. The NR2E1 mini-promoter element may have a nucleic acid sequence substantially similar in sequence and function to SEQ ID NO: 1. The NR2E1 basal promoter element may have a nucleic acid sequence substantially similar in sequence and function to SEQ ID NO: 2. The NR2E1 regulatory element may have a nucleic acid sequence substantially similar in sequence and function to SEQ ID NO: 3, 4, and/or 5. The disease or condition may be chosen from: retinal diseases, retinal degeneration, retinal damage, blindness, macular degeneration, retinitis pigmentosa, inherited retinal genetic diseases, diabetic retinopathy, cone rod dystrophy, hypertensive/diabetic retinopathy. The therapeutic or beneficial compound may be a light-sensitive compound, for instance rhodopsin, channel rhodopsin, etc.

### SHORT DESCRIPTION OF FIGURES

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1. DNA expression vector (pEMS1313) into which NR2E1 promoter elements were inserted for expression studies operably links the NR2E1 MiniPromoter to the lac Z reporter gene. The construct also contains the HPRT genomic targeting sequence, an ampicillin resistance gene (AmpR) for screening, and a transcriptional termination sequence (SV40 polyA), as well as other elements necessary for vector replication and gene expression.

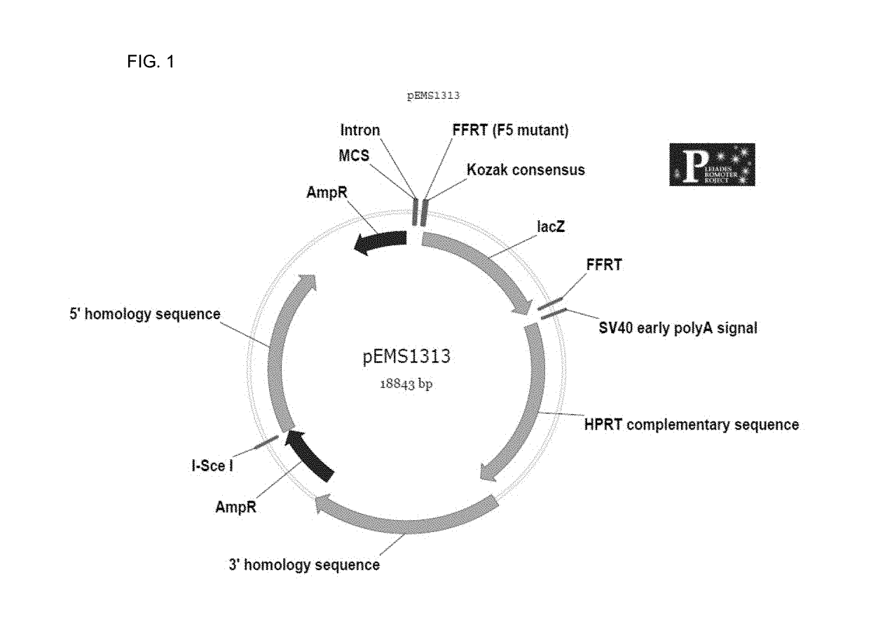

FIG. 2A-2C. Expression of NR2E1-based MiniPromoter Ple264 in the Müller glia of the mouse retina. (A,B) The vEMS23 ssAAV9 virus was injected intravenously into P0 mice containing the Gt(ROSA26)Sor^{tm1Sor} locus. This virus contains the Ple264 MiniPromoter, based on NR2E1 RRs, driving expression of the iCre recombinase. Upon expression, recombination of the ROSA26 locus via iCre results in expression of β-galactosidase (lacZ). Eyes were harvested at P21, stained for lacZ activity using the X-gal substrate (blue product) and counter-stained with neutral red to visualize cell nuclei. Expression was observed in the ganglion cell layer, but predominantly in cell bodies of the inner nuclear layer with long processes extending the span of the neural retina consistent with, and unique to, Müller glial cells. Three animals are shown. (FIG. 2A) Whole mount view down onto the innermost part of the retina. (FIG. 2B) Two animals with a staining in the Müller glia. Insets show a magnified view. (FIG. 2C) The Ple264 MiniPromoter driving iCre was knocked-in at the Hprt locus in mouse embryonic stem cells. Germline mice were obtained and bred to the Ai14 reporter mouse to detect MiniPromoter expression, as determined by tdTomato fluorescent protein. Adult mouse eyes were harvested and visualized using a laser confocal microscope. Throughout the retina characteristic Müller glia staining was observed, with cell bodies located in the middle of the INL and processes extending to the GCL (end-feet) and to the edge of the ONL. GCL, ganglion cell layer; INL, inner nuclear layer; ONL, outer nuclear layer; P#, post-natal day #; RRs, regulatory regions; ssAAV9, single-stranded adeno-associated virus serotype 9.

FIG. 3A-3C. Expression of NR2E1-based Mini-Promoter Ple140 (SEQ ID NO: 6) in mouse brain. Coronal 1-mm sections of the brain were stained in X-gal solution overnight to detect lacZ reporter gene activity (blue). FIG. 3A, Overview image shows expression in the ventral aspects of the hypothalamus. FIG. 3B, Close-up view of the hypothalamus. FIG. 3C, Some scattered staining is also observed in the amygdala.

FIG. 4. pEMS1980 virus backbone vector used for the Ple264 construct.

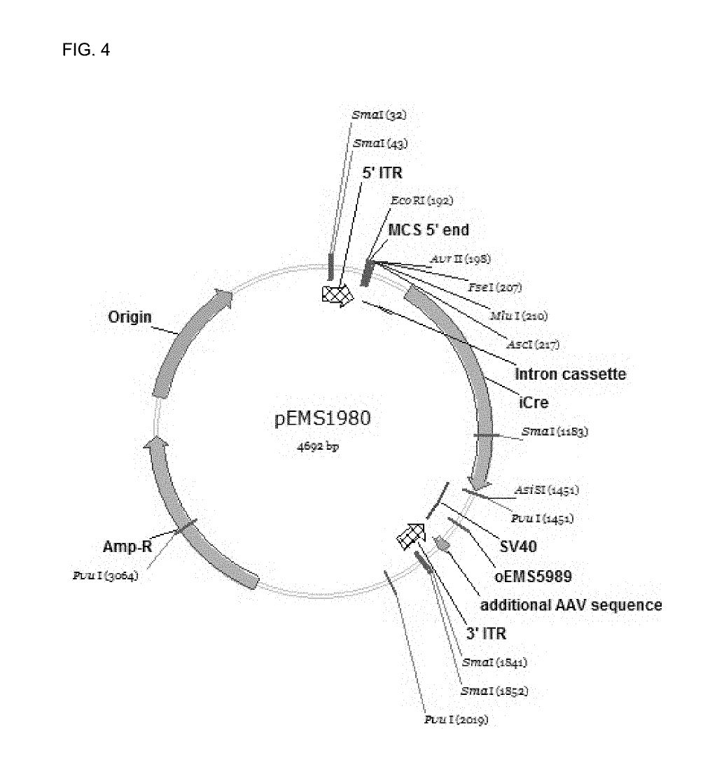

FIG. 5. Diagram of the mouse allele for Ple264 knocked-in at the Hprt locus.

### DETAILED DESCRIPTION

The compositions of the present invention include novel polynucleotides comprising NR2E1 promoter elements (also referred to herein as NR2E1 mini-promoters) as well as novel expression vectors comprising said NR2E1 promoter elements (or mini-promoters). The present invention also includes various methods utilizing these novel NR2E1 promoter (or mini-promoter) elements or expression vectors.

The term ‘NR2E1’ refers to the gene, which encodes the NR2E1 protein, also referred to as nuclear receptor subfamily 2, group E, member 1. The human homolog of NR2E1 is encoded by the human gene identified as EntrezGene #7101 and is located at chromosomal location 6q21. The protein encoded by human NR2E1 has the Protein Accession # Q9Y466.1, however other protein accession numbers may also be assigned to this protein. NR2E1 may also include other isoforms and/or splice variants. Other mammalian NR2E1 homologs may include but are not limited to: Rattus norvegicus (EntrezGene #684085), Mus musculus (EntrezGene #21907).

The term ‘promoter’ refers to the regulatory DNA region which controls transcription or expression of a gene and which can be located adjacent to or overlapping a nucleotide or region of nucleotides at which RNA transcription is initiated. A promoter contains specific DNA sequences which bind protein factors, often referred to as transcription factors, which facilitate binding of RNA polymerase to the DNA leading to gene transcription. A ‘basal promoter’, also referred to as a ‘core promoter’, usually means a promoter which contains all the basic necessary elements to promote transcriptional expression of an operably linked polynucleotide. Eukaryotic basal promoters typically, though not necessarily, contain a TATA-box and/or a CAAT box. A ‘NR2E1 basal promoter’, in the context of the present invention and as used herein, is a nucleic acid compound having a sequence with at least 65%, at least 70%, at least 80%, at least 85%, at least 90%, at least 95%, or at least 99% similarity to SEQ ID NO: 2.

A promoter may also include ‘regulatory elements’ that influence the expression or transcription by the promoter. Such regulatory elements encode specific DNA sequences which bind other factors, which may include but are not limited to enhancers, silencers, insulators, and/or boundary elements. A ‘NR2E1 regulatory element’, in the context of the present invention and as used herein, is a nucleic acid compound having a sequence with at least 65%, at least 70%, at least 80%, at least 85%, at least 90%, at least 95%, or at least 99% similarity to SEQ ID NO: 3, 4 and/or 5. The present invention provides, in certain embodiments as described herein, different promoters of the NR2E1 gene. In some embodiments, the NR2E1 promoter comprises a NR2E1 regulatory element operably linked to a NR2E1 basal promoter.

An NR2E1 mini-promoter typically includes a basal promoter as described above and one or more regulatory elements as described above. A “NR2E1 mini-promoter”, in the context of the present invention and as used herein, includes a nucleic acid compound having a sequence with at least 65%, at least 70%, at least 80%, at least 85%, at least 90%, at least 95%, or at least 99% similarity to SEQ ID NO: 1. In some embodiments the mini-promoter lacks regulatory elements that are present in SEQ ID NO:6 and absent in SEQ ID NO:1.

The term ‘operably linked’, in the context of the present invention, means joined in such a fashion as to work together to allow transcription. In some embodiments of the invention, two polynucleotide sequences may be operably linked by being directly linked via a nucleotide bond. In this fashion, the two operably linked elements contain no intervening sequences and in being joined are able to direct transcription of an expression sequence. In other embodiments of the invention, two elements may be operably linked by an intervening compound, for instance a polynucleotide sequence of variable length. In such a fashion, the operably linked elements, although not directly juxtaposed, are still able to direct transcription of an expression sequence. Thus, according to some embodiments of the invention, one or more promoter elements may be operably linked to each other, and additionally be operably linked to a downstream expression sequence, such that the linked promoter elements are able to direct expression of the downstream expression sequence.

The term ‘mini-promoter’ refers to a promoter in which certain promoter elements are selected from an endogenous full length promoter for a gene, usually in such a fashion as to reduce the overall size of the promoter compared to the native sequence. For example, after identification of critical promoter elements, using one or more of various techniques, the native sequences that intervene between identified elements may be partially or completely removed. Other non-native sequences may optionally be inserted between the identified promoter elements. Promoter sequences such as enhancer elements may have an orientation that is different from the native orientation—for example, a promoter element may be inverted, or reversed, from its native orientation. Alternatively, selecting a minimal basal promoter that is sufficient to drive expression in particular cells or tissues may also be desirable. Since promoter elements that impact expression patterns are known to be distributed over varying distances of the proximal and/or distal endogenous promoter, it is a non-trivial task to identify a mini-promoter comprising a minimal basal promoter and optional regulatory regions that will adequately express in the desired cell or tissue types. A mini-promoter may provide certain advantages over native promoter conformations. For example, the smaller size of the mini-promoter may allow easier genetic manipulation, for example in the design and/or construction of expression vectors or other recombinant DNA constructs. In addition, the smaller size may allow easier insertion of DNA constructs into host cells and/or genomes, for example via transfection, transformation, etc. Other advantages of mini-promoters are apparent to one of skill in the art. In some embodiments of the invention, there are thus provided novel NR2E1 mini-promoters comprising a NR2E1 regulatory element operably linked in a non-native conformation to a NR2E1 basal promoter. In general the spacing between the NR2E1 regulatory element and the NR2E1 basal promoter is not more than about 15 KB, generally not more than about 10 KB, usually not more than about 1 KB, more often not more than about 500 nt, and may be not more than about 100 nt, down to a direct joining of the two sequences. In other embodiments, there is provided a minimal NR2E1 basal promoter.

The term ‘expressible sequence’ refers to a polynucleotide composition which is operably linked to a promoter element such that the promoter element is able to cause transcriptional expression of the expression sequence. An expressible sequence is typically linked downstream, on the 3′-end of the promoter element(s) in order to achieve transcriptional expression. The result of this transcriptional expression is the production of an RNA macromolecule. The expressed RNA molecule may encode a protein and may thus be subsequently translated by the appropriate cellular machinery to produce a polypeptide protein molecule. In some embodiments of the invention, the expression sequence may encode a reporter protein. Alternately, the RNA molecule may be an antisense, RNAi or other non-coding RNA molecule, which may be capable of modulating the expression of specific genes in a cell, as is known in the art.

The term ‘RNA’ as used in the present invention includes full-length RNA molecules, which may be coding or non-coding sequences, fragments, and derivatives thereof. For example, a full-length RNA may initially encompass up to about 20 Kb or more of sequence, and frequently will be processed by splicing to generate a small mature RNA. Fragments, RNAi, miRNA and anti-sense molecules may be smaller, usually at least about 18 nt. in length, at least about 20 nt in length, at least about 25 nt. in length, and may be up to about 50 nt. in length, up to about 100 nt in length, or more. RNA may be single stranded, double stranded, synthetic, isolated, partially isolated, essentially pure or recombinant. RNA compounds may be naturally occurring, or they may be altered such that they differ from naturally occurring RNA compounds. Alterations may include addition, deletion, substitution or modification of existing nucleotides. Such nucleotides may be either naturally occurring, or non-naturally occurring nucleotides. Alterations may also involve addition or insertion of non-nucleotide material, for instance at the end or ends of an existing RNA compound, or at a site that is internal to the RNA (ie. between two or more nucleotides).

The term ‘nucleic acid’ as used herein includes any nucleic acid, and may be a deoxyribonucleotide or ribonucleotide polymer in either single or double-stranded form. A ‘polynucleotide’ or ‘nucleotide polymer’ as used herein may include synthetic or mixed polymers of nucleic acids, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e. g., phosphorothioates, phosphorodithioates, etc.), pendent moieties (e.g., polypeptides), and modified linkages (e.g., alpha anomeric polynucleotides, etc.). Also included are synthetic molecules that mimic polynucleotides in their ability to bind to a designated sequence via hydrogen bonding and other chemical interactions.

A ‘purine’ is a heterocyclic organic compound containing fused pyrimidine and imidazole rings, and acts as the parent compound for purine bases, adenine (A) and guanine (G). ‘Nucleotides’ are generally a purine (R) or pyrimidine (Y) base covalently linked to a pentose, usually ribose or deoxyribose, where the sugar carries one or more phosphate groups. Nucleic acids are generally a polymer of nucleotides joined by 3′ 5′ phosphodiester linkages. As used herein ‘purine’ is used to refer to the purine bases, A and G, and more broadly to include the nucleotide monomers, deoxyadenosine-5′-phosphate and deoxyguanosine-5′-phosphate, as components of a polynucleotide chain. A ‘pyrimidine’ is a single-ringed, organic base that forms nucleotide bases, such as cytosine (C), thymine (T) and uracil (U). As used herein ‘pyrimidine’ is used to refer to the pyrimidine bases, C, T and U, and more broadly to include the pyrimidine nucleotide monomers that along with purine nucleotides are the components of a polynucleotide chain.

It is within the capability of one of skill in the art to modify the sequence of a promoter nucleic acid sequence, e.g. the provided basal promoter and regulatory sequences, in a manner that does not substantially change the activity of the promoter element, i.e. the transcription rate of an expressible sequence operably linked to a modified promoter sequence is at least about 65% the transcription rate of the original promoter, at least about 75% the transcription rate of the original promoter sequence, at least about 80%, at least about 90%, at least about 95%, at least about 99%, or more. Such modified sequences would be considered to be ‘functionally similar’ or to have ‘functional similarity’ or ‘substantial functional similarity’ to the unmodified sequence. Such modifications may include insertions, deletions which may be truncation of the sequence or internal deletions, or substitutions. The level of sequence modification to an original sequence will determine the ‘sequence similarity’ of the original and modified sequences. Modification of the promoter elements of the present invention in a fashion that does not significantly alter transcriptional activity, as described above would result in sequences with ‘substantial sequence similarity’ to the original sequence i.e. the modified sequence has a nucleic acid composition that is at least about 65% similar to the original promoter sequence, at least about 75% similar to the original promoter sequence, at least about 80%, at least about 90%, at least about 95%, at least about 99%, or more similar to the original promoter sequence.

Thus, mini-promoter elements which have substantial functional and/or sequence similarity are herein described and are within the scope of the invention.

An ‘RNA interference molecule’, or ‘RNA interference sequence’ as defined herein, may include, but is not limited to, an antisense RNA molecule, a microRNA molecule or a short hairpin RNA (shRNA) molecule. Typically, RNA interference molecules are capable of target-specific modulation of gene expression and exert their effect either by mediating degradation of the mRNA products of the target gene, or by preventing protein translation from the mRNA of the target gene. The overall effect of interference with mRNA function is modulation of expression of the product of a target gene. This modulation can be measured in ways which are routine in the art, for example by Northern blot assay or reverse transcriptase PCR of mRNA expression, Western blot or ELISA assay of protein expression, immunoprecipitation assay of protein expression, etc.

An ‘antisense RNA molecule’, as used herein, is typically a single stranded RNA compound which binds to complementary RNA compounds, such as target mRNA molecules, and blocks translation from the complementary RNA compounds by sterically interfering with the normal translational machinery. Specific targeting of antisense RNA compounds to inhibit the expression of a desired gene may design the antisense RNA compound to have a homologous, complementary sequence to the desired gene. Perfect homology is not necessary for inhibition of expression. Design of gene specific antisense RNA compounds, including nucleotide sequence selection and additionally appropriate alterations, are known to one of skill in the art.

The term ‘microRNA molecule’, ‘microRNA’ or ‘miRNA’, as used herein, refers to single-stranded RNA molecules, typically of about 21-23 nucleotides in length, which are capable of modulating gene expression. Mature miRNA molecules are partially complementary to one or more messenger RNA (mRNA) molecules, and their main function is to downregulate gene expression. Without being bound by theory, miRNAs are first transcribed as primary transcripts or pri-miRNA with a cap and poly-A tail and processed to short, 70-nucleotide stem-loop structures known as pre-miRNA in the cell nucleus. This processing is performed in animals by a protein complex known as the Microprocessor complex, consisting of the nuclease Drosha and the double-stranded RNA binding protein Pasha. These pre-miRNAs are then processed to mature miRNAs in the cytoplasm by interaction with the endonuclease Dicer, which also initiates the formation of the RNA-induced silencing complex (RISC). When Dicer cleaves the pre-miRNA stem-loop, two complementary short RNA molecules are formed, but only one is integrated into the RISC complex. This strand is known as the guide strand and is selected by the argonaute protein, the catalytically active RNase in the RISC complex, on the basis of the stability of the 5′ end. The remaining strand, known as the anti-guide or passenger strand, is degraded as a RISC complex substrate. After integration into the active RISC complex, miRNAs base pair with their complementary mRNA molecules and induce mRNA degradation by argonaute proteins, the catalytically active members of the RISC complex. Animal miRNAs are usually complementary to a site in the 3′ UTR whereas plant miRNAs are usually complementary to coding regions of mRNAs.

The term ‘short hairpin RNA’ or ‘shRNA’ refers to RNA molecules having an RNA sequence that makes a tight hairpin turn that can be used to silence gene expression via RNA interference. The shRNA hairpin structure is cleaved by the cellular machinery into siRNA, which is then bound to the RNA-induced silencing complex (RISC). This complex binds to and cleaves mRNAs which match the siRNA that is bound to it. shRNA is transcribed by RNA Polymerase III whereas miRNA is transcribed by RNA Polymerase II. Techniques for designing target specific shRNA molecules are known in the art.

An ‘expression vector’ is typically a nucleic acid molecule which may be integrating or autonomous, (i.e. self-replicating), and which contains the necessary components to achieve transcription of an expressible sequence in a target cell, when introduced into the target cell. Expression vectors may include plasmids, cosmids, phage, YAC, BAC, mini-chromosomes, viruses, e.g. retroviruses, adenovirus, lentivirus, SV-40, and the like; etc. Many such vectors have been described in the art and are suitable for use with the promoters of the present invention. Expression vectors of the present invention include a promoter as described herein, operably linked to an expressible sequence, which may also be optionally operably linked to a transcription termination sequence, such as a polyadenylation sequence. The expression vector optionally contains nucleic acid elements which confer host selectivity, elements that facilitate replication of the vector, elements that facilitate integration of the vector into the genome of the target cell, elements which confer properties, for example antibiotic resistance, to the target cell which allow selection or screening of transformed cells and the like. Techniques and methods for design and construction of expression vectors are well known in the art.

It may be desirable, when driving expression of an expressible sequence with a particular promoter system to have the expression occur in a stable and consistent manner. A factor that has been shown to affect expression is the site of integration of an expression vector or construct into the genome of the target cell, sometimes called ‘position effects’. Such position effects may be caused by, for example, local chromatin structure which affects expression of sequences from that region of the genome. One method to control for position effects when integrating an expression vector or construct into the genome of a target cell is to include a ‘genomic targeting sequence’ in the vector or construct that directs integration of the vector or construct to a specific genomic site. As an example, the hypoxanthine phosphoribosyltransferase (HPRT) gene has been used successfully for this purpose (Bronson, Plaehn et al. 1996; Jasin, Moynahan et al. 1996). The HPRT gene has additional advantages as a genomic targeting sequence, for instance its concomitant use as a selectable marker system. Other genomic targeting sequences that may be useful in the present invention are described in the art, for instance (Jasin, Moynahan et al. 1996; van der Weyden, Adams et al. 2002). The genomic targeting signals as described herein are useful in certain embodiments of the present invention.

Introduction of nucleic acids or expression vectors into cells may be accomplished using techniques well known in the art, for example microinjection, electroporation, particle bombardment, or chemical transformation, such as calcium-mediated transformation, as described for example in Maniatis et al. 1982, Molecular Cloning, A laboratory Manual, Cold Spring Harbor Laboratory or in Ausubel et al. 1994, Current protocols in molecular biology, Jolm Wiley and Sons.

In certain embodiments of the invention, there are provided methods of treatment using the nucleic acids or expression vectors, for instance for gene therapy applications. The nucleic acids or expression vectors of the present invention may be administered in isolation, or may be linked to or in combination with tracer compounds, liposomes, carbohydrate carriers, polymeric carriers or other agents or excipients as will be apparent to one of skill in the art. In an alternate embodiment, such compounds may comprise a medicament, wherein such compounds may be present in a pharmacologically effective amount.

The term ‘medicament’ as used herein refers to a composition that may be administered to a patient or test subject and is capable of producing an effect in the patient or test subject. The effect may be chemical, biological or physical, and the patient or test subject may be human, or a non-human animal, such as a rodent or transgenic mouse, or a dog, cat, cow, sheep, horse, hamster, guinea pig, rabbit or pig. The medicament may be comprised of the effective chemical entity alone or in combination with a pharmaceutically acceptable excipient.

The term ‘pharmaceutically acceptable excipient’ may include any and all solvents, dispersion media, coatings, antibacterial, antimicrobial or antifungal agents, isotonic and absorption delaying agents, and the like that are physiologically compatible. An excipient may be suitable for intravenous, intraperitoneal, intramuscular, subcutaneous, intrathecal, intraocular, topical or oral administration. An excipient may include sterile aqueous solutions or dispersions for extemporaneous preparation of sterile injectable solutions or dispersion. Use of such media for preparation of medicaments is known in the art.

The nucleic acids or expression vectors of the present invention may be administered to a subject using a viral delivery system. For instance, the nucleic acids may be inserted into a viral vector using well known recombinant techniques. The subsequent viral vector may then be packaged into a virus, such as adenovirus, lentivirus, attenuated virus, adeno-associated virus (AAV), and the like. Viral delivery for gene therapy applications is well known in the art. There exist a variety of options for viruses suitable for such delivery, which may also involve selecting an appropriate viral serotype for delivery and expression in an appropriate tissue.

Compositions or compounds according to some embodiments of the invention may be administered in any of a variety of known routes. Examples of methods that may be suitable for the administration of a compound include orally, intravenous, inhalation, intramuscular, subcutaneous, topical, intraperitoneal, intra-ocular, intra-rectal or intra-vaginal suppository, sublingual, and the like. The compounds of the present invention may be administered as a sterile aqueous solution, or may be administered in a fat-soluble excipient, or in another solution, suspension, patch, tablet or paste format as is appropriate. A composition comprising the compounds of the invention may be formulated for administration by inhalation. For instance, a compound may be combined with an excipient to allow dispersion in an aerosol.

Examples of inhalation formulations will be known to those skilled in the art. Other agents may be included in combination with the compounds of the present invention to aid uptake or metabolism, or delay dispersion within the host, such as in a controlled-release formulation. Examples of controlled release formulations will be known to those of skill in the art, and may include microencapsulation, embolism within a carbohydrate or polymer matrix, and the like. Other methods known in the art for making formulations are found in, for example, “Remington's Pharmaceutical Sciences”, (19th edition), ed. A. Gennaro, 1995, Mack Publishing Company, Easton, Pa.

The dosage of the compositions or compounds of some embodiments of the invention may vary depending on the route of administration (oral, intravenous, inhalation, or the like) and the form in which the composition or compound is administered (solution, controlled release or the like). Determination of appropriate dosages is within the ability of one of skill in the art. As used herein, an ‘effective amount’, a ‘therapeutically effective amount’, or a ‘pharmacologically effective amount’ of a medicament refers to an amount of a medicament present in such a concentration to result in a therapeutic level of drug delivered over the term that the drug is used. This may be dependent on mode of delivery, time period of the dosage, age, weight, general health, sex and diet of the subject receiving the medicament. Methods of determining effective amounts are known in the art. It is understood that it could be potentially beneficial to restrict delivery of the compounds of the invention to the target tissue or cell in which protein expression. It is also understood that it may be desirable to target the compounds of the invention to a desired tissue or cell type. The compounds of the invention may thus be coupled to a targeting moiety. The compounds of the invention may be coupled to a cell uptake moiety. The targeting moiety may also function as the cell uptake moiety.

NR2E1 Mini-Promoters

The present invention herein provides novel NR2E1 mini-promoter sequences which are capable of effecting transcriptional expression in a spatial and temporal fashion in the brain and/or eye. Certain NR2E1 mini-promoters of the invention comprise minimal NR2E1 promoter elements joined in a non-native configuration, thus providing advantageous characteristics. Also provided are novel expression vector compositions comprising NR2E1 mini-promoters which allow consistent specific spatiotemporal transcription of expression sequences. Also provided are novel methods utilizing these NR2E1 mini-promoters and expression vectors.

The NR2E1 promoters of the invention, as described herein, are referred to as ‘mini-promoters’ to reflect the fact that the mini-promoters comprise minimal NR2E1 promoter elements sufficient to drive expression, and that may also be joined by non-native sequences. In this context, the native intervening sequences may have been partially or completely removed, and optionally may have been replaced with non-native sequences. Furthermore, the natural spatial arrangement of elements may be altered, such that downstream promoter elements (in natural conformation) are moved upstream (in non-native conformation). In such a fashion, the natural spacing of the promoter elements, for instance a human NR2E1 regulatory element corresponding to SEQ ID NO: 3, 4, and/or 5, and the human NR2E1 basal promoter element corresponding to SEQ ID NO: 2, or sequences with substantial functional and/or sequence equivalence, is altered. Additionally, the orientation of the different promoter elements may be altered—for instance the regulatory element corresponding to SEQ ID NO: 3, 4, and/or 5 may be inverted relative to the basal promoter element corresponding to SEQ ID NO: 2. An advantage of such non-native mini-promoters is that the removal of native intervening sequences reduces the size of the mini-promoter while maintaining the functional activity of the promoter, thus improving the utility of the mini-promoter for various applications. Furthermore, the inversion of an enhancer/promoter element may allow retention of the enhancer properties without causing alternate promoter activity.

The inventors have demonstrated, as illustrated in the non-limiting Working Examples, that a human NR2E1 mini-promoter having a sequence corresponding to SEQ ID NO: 1 (also referred to in the Working Examples as Ple264) is capable of directing expression of an expressible sequence which is operably linked downstream of the NR2E1 promoter in specific cell types in different regions of the brain and/or eye, in contrast to the mini-promoter of SEQ ID NO:6. SEQ ID NO:1 is comprised of 3 human NR2E1 regulatory elements (corresponding to SEQ ID NOs: 3, 4, and 5) operably linked in a non-native conformation to a human NR2E1 basal promoter having a nucleic acid sequence corresponding to SEQ ID NO: 2, The NR2E1 regulatory elements (SEQ ID NO'S: 3-5) and NR2E1 basal promoter element (SEQ ID NO: 2) have sequences which are identical to those found in the human NR2E1 gene. It is within the skill of one in the art to locate and determine these relative positions based on published sequence information for this gene, for instance found in the GenBank or PubMed public databases. It is understood that these genomic coordinates and relative positions are provided for the purposes of context, and that if any discrepancies exist between published sequences and the sequence listings provided herein, then the sequence listings shall prevail.

Promoters of the present invention may be modified with respect to the native regulatory and/or native basal promoter sequence. In general, such modifications will not change the functional activity of the promoter with respect to cell-type selectivity; and to the rate of transcription in cells where the promoter is active. The modified promoter provide for a transcription rate of an expressible sequence operably linked to a modified promoter sequence that is at least about 75% the transcription rate of the promoter sequence of SEQ ID NO: 1, at least about 80%, at least about 90%, at least about 95%, at least about 99%, or more. Methods of assessing promoter strength and selectivity are known in the art, including, for example, expression of a reporter sequence in a cell in vivo or in vitro, and quantitating the reporter activity.

Modifications of interest include deletion of terminal or internal regions, and substitution or insertion of residues. The spacing of conserved sequences may be the same as the native spacing, or it may be different than the native spacing. The order of the conserved sequences may be the same as the native order or the sequences may be rearranged. Sequences set forth in SEQ ID NO: 1 that are not conserved may be deleted or substituted, usually modifications that retain the spacing between conserved sequences is preferred. In general the spacing between the regulatory element and the basal promoter is not more than about 10 KB, generally not more than about 1 KB, usually not more than about 500 nt, and may be not more than about 100 nt, down to a direct joining of the two sequences.

In one embodiment of the invention, there is provided an isolated nucleic acid fragment comprising a NR2E1 mini-promoter, wherein the NR2E1 mini-promoter comprises one or more NR2E1 regulatory elements operably linked in a non-native conformation to a NR2E1 basal promoter. The NR2E1 mini-promoter may have a nucleic acid sequence which is substantially similar in sequence and function to SEQ ID NO: 1. The NR2E1 basal promoter may have a nucleic acid sequence which is substantially similar in sequence and function to SEQ ID NO: 2. The NR2E1 regulatory element may have a nucleic acid sequence which is substantially similar in sequence and function to SEQ ID NO: 3, 4, and/or 5. The NR2E1 mini-promoters may further be operably linked to an expressible sequence, e.g. reporter genes, genes encoding a polypeptide of interest, regulatory RNA sequences such as miRNA, siRNA, anti-sense RNA, etc., and the like. Reporter gene sequences include, for example luciferase, beta-galactosidase, green fluorescent protein, enhanced green fluorescent protein, and the like as known in the art. The expressible sequence may encode a protein of interest, for example a therapeutic protein, receptor, antibody, growth factor, transcription factor, and the like. The expressible sequence may encode an RNA interference molecule.

It is an object of the present invention to provide means of expressing a gene, protein, RNA interference molecule or the like in a cell, tissue or organ. As such, the inventors thus provide novel expression vectors comprising NR2E1 mini-promoters which are capable of accomplishing this task. In one embodiment, there is provided an expression vector comprising a NR2E1 mini-promoter, wherein the NR2E1 mini-promoter comprises one or more NR2E1 regulatory element operably linked in a non-native conformation to an NR2E1 basal promoter. The NR2E1 mini-promoter may have a nucleic acid sequence which is substantially similar in sequence and function to SEQ ID NO: 1. The NR2E1 basal promoter may have a nucleic acid sequence which is substantially similar in sequence and function to SEQ ID NO: 2. The NR2E1 regulatory element may have a nucleic acid sequence which is substantially similar in sequence and function to SEQ ID NO: 3, 4, and/or 5. The NR2E1 mini-promoter may further be operably linked to an expressible sequence, e.g. reporter genes, genes encoding a polypeptide of interest, regulatory RNA sequences such as miRNA, siRNA, anti-sense RNA, etc., and the like. Reporter gene sequences include, for example luciferase, beta-galactosidase, green fluorescent protein, enhanced green fluorescent protein, and the like as known in the art. The expressible sequence may encode a protein of interest, for example a therapeutic protein, receptor, antibody, growth factor, transcription factor, and the like. The expressible sequence may encode an RNA interference molecule. The expression vector may further comprise a genomic targeting sequence. The genomic targeting sequence may be HPRT, e.g. human HPRT, mouse HPRT, etc.

The inventors have herein demonstrated that expression vectors comprising novel NR2E1 mini-promoter elements are capable of directing transcription of an expression sequence in specific cell types, for instance in Müller glia cells in the retina (eye) or in neuronal cells in the brain. In one embodiment of the invention, there is thus provided a method for expressing a gene, protein, RNA interference molecule or the like in a cell, the method comprising introducing into the cell an expression vector comprising an NR2E1 mini-promoter element, wherein the NR2E1 mini-promoter element comprises one or more NR2E1 regulatory element operably linked in a non-native conformation to a NR2E1 basal promoter element. Cells of interest include, without limitation, cells of the peripheral or central nervous system and progenitors thereof, e.g. embryonic stem cells, neural stem cells, neurons, glial cells, astrocytes, etc; and/or cells in the eye and progenitors thereof, e.g. retinal cells, Müller glial cells etc. The NR2E1 mini-promoter may have a nucleic acid sequence which is substantially similar in sequence and function to SEQ ID NO: 1. The NR2E1 basal promoter may have a nucleic acid sequence which is substantially similar in sequence and function to SEQ ID NO: 2. The NR2E1 regulatory element may have a nucleic acid sequence which is substantially similar in sequence and function to SEQ ID NO: 3, 4, and/or 5. The NR2E1 mini-promoter may further be operably linked to an expressible sequence, e.g. reporter genes, genes encoding a polypeptide of interest, regulatory RNA sequences such as miRNA, siRNA, anti-sense RNA, etc., and the like. Reporter gene sequences include, for example luciferase, beta-galactosidase, green fluorescent protein, enhanced green fluorescent protein, and the like as known in the art. The expressible sequence may encode a protein of interest, for example a therapeutic protein, receptor, antibody, growth factor, transcription factor, and the like. The expressible sequence may encode an RNA interference molecule. The expression vector may thus further comprise a genomic targeting sequence. The genomic targeting sequence may be HPRT.

In one embodiment of the invention, there is provided a method for identifying or labeling a cell, the method comprising introducing into the cell an expression vector comprising an NR2E1 mini-promoter element, wherein the NR2E1 mini-promoter element comprises one or more NR2E1 regulatory element operably linked in a non-native conformation to a NR2E1 basal promoter element, and wherein the expressible sequence comprises a reporter gene. The NR2E1 mini-promoter element may have a nucleic acid sequence substantially similar in sequence and function to SEQ ID NO: 1. The NR2E1 basal promoter element may have a nucleic acid sequence substantially similar in sequence and function to SEQ ID NO: 2. The NR2E1 regulatory element may have a nucleic acid sequence substantially similar in sequence and function to SEQ ID NO: 3, 4, and/or 5. The inventors have demonstrated that expression vectors comprising certain human NR2E1 promoter elements are capable of expression in specific regions of the brain and eye, most notably retinal Müller glial cells in the eye. In some embodiments, the cell is a peripheral or central nervous system cell or progenitors thereof, including, without limitation, embryonic stem cells, neural stem cells, glial cell, neuronal cells, astrocytes, and the like. In some embodiments, the cell is a cell of the eye and progenitors thereof, including without limitation retinal cells, retinal Müller glial cells, and the like. Reporter gene sequences include, for example luciferase, beta-galactosidase, green fluorescent protein, enhanced green fluorescent protein, and the like as known in the art. The expressible sequence may encode a protein of interest, for example a therapeutic protein, receptor, antibody, growth factor, transcription factor, RNA interference molecule and the like.

In further embodiments of the invention, there is provided a method for monitoring or tracking the development or maturation of a cell, the method comprising: 1) introducing into the cell a expression vector comprising an NR2E1 mini-promoter element operably linked to an expressible sequence, wherein the NR2E1 mini-promoter element comprises one or more NR2E1 regulatory elements operably linked in a non-native conformation to an NR2E1 basal promoter element, and wherein the expressible sequence comprises a reporter gene; and 2) detecting the expression of the reporter gene in the cell of in progeny of the cell as a means of determining the lineage, identity or developmental state of the cell or cell progeny. The NR2E1 mini-promoter element may have a nucleic acid sequence substantially similar in sequence and function to SEQ ID NO: 1. The NR2E1 basal promoter element may have a nucleic acid sequence substantially similar in sequence and function to SEQ ID NO: 2. The NR2E1 regulatory element may have a nucleic acid sequence substantially similar in sequence and function to SEQ ID NO: 3, 4 and/or 5. In such a fashion, one may be able to follow the development of a parent cell as it differentiates into more mature cells. As an example, one could introduce an expression vector comprising the aforementioned NR2E1 mini-promoter elements into a pluripotent stem cell, monitor the expression of the reporter gene that is being expressed by the NR2E1 promoter elements during the maturation and differentiation of the stem cell and thus determine the state of maturation, for instance in the differentiation of the pluripotent stem cell into a specific brain or retinal cell type. The inventors have demonstrated that the NR2E1 mini-promoter elements described herein direct transcriptional expression in certain brain and retinal cell types, and so detection of reporter gene expression in a cell would thus be indicative of the cellular identity of the cell as being a certain type of brain or retinal cell.

The inventors have herein demonstrated that certain NR2E1 mini-promoter elements of the present invention are capable of driving expression in retinal Müller glial cells. This surprising expression pattern provides additional methods of use for these mini-promoter elements. For instance, it may be desirable to utilize the NR2E1 mini-promoters of the present invention in a gene therapy or cell therapy application wherein the NR2E1 mini-promoters are utilized to drive expression of a therapeutic or beneficial compound, such as a protein, in retinal Müller glial cells. In such a way, the therapeutic or beneficial compound may be useful for a disease or condition that involves such retinal cells, or which may be improved by expression of the therapeutic or beneficial compound in those cells. In certain embodiments of the invention, there is thus provided a method of treatment of a subject having a disease or condition of the eye, the method comprising administering to the subject a therapeutically effective dose of a composition comprising an NR2E1 mini-promoter element, wherein the NR2E1 mini-promoter element comprises one or more NR2E1 regulatory element operably linked in a non-native conformation to an NR2E1 basal promoter element. The NR2E1 mini-promoter element may have a nucleic acid sequence substantially similar in sequence and function to SEQ ID NO: 1. The NR2E1 basal promoter element may have a nucleic acid sequence substantially similar in sequence and function to SEQ ID NO: 2. The NR2E1 regulatory element may have a nucleic acid sequence substantially similar in sequence and function to SEQ ID NO: 3, 4, and/or 5. The disease or condition may be chosen from: retinal diseases, retinal degeneration, retinal damage, blindness, macular degeneration, retinitis pigmentosa, inherited retinal genetic diseases, diabetic retinopathy, cone rod dystrophy, hypertensive/diabetic retinopathy. The therapeutic or beneficial compound may be a light-sensitive compound, for instance rhodopsin, channel rhodopsin, etc.

The inventors herein further describe the present invention by way of the following non-limiting examples:

### WORKING EXAMPLES

General Methods

Virus Generation and Analysis

Virus Production

The Ple264 (NR2E1) MiniPromoter was generated by direct synthesis by DNA2.0 (Menlo Park, Calif., USA) (SEQ ID NO: 1). Ple264 was cloned into the pEMS1980 backbone, containing the iCre reporter, using AvrII and AscI restriction enzymes. One μg of pEMS1981 plasmid containing the Ple264 MiniPromoter was prepared by miniprep and sent to the Vector Core at the University of Pennsylvania (Philadelphia, Pa., USA) to be made into AAV9 serotype virus.

Virus Injection

B6-Gt(ROSA26)^{tm1Sor} females were crossed to 129-Gt(ROSA26)^{tm1Sor} to yield hybrid F1 homozygous pups for injecting virus. Plug checks were performed on the females such that the day of birth could be accurately estimated. P0 pups were used for virus injections. If the female gave birth in the morning, virus was injected in the afternoon. If she gave birth in the afternoon, virus was injected the next morning. A standard injection into the superficial temporal vein of a newborn pup was performed using 1×10¹³ GC/mL (genome copies per milliliter) virus in a total volume of 50 μL (in PBS) with a 30 gauge needle and a 1 cc syringe. After injections, pups were tattooed for identification and returned to their cage.

Harvesting of Animals

Virus-injected mice were harvested at P21 or P56 (post-natal day 21 or 56). Animals were given a lethal dose of avertin injected intraperitoneally. Thereafter perfusion with 1×PBS for 2 minutes and 4% PFA/PBS for 8 minutes was performed. Tissues were harvested and post-fixed for 1 hour at 4° C. The tissues were then stored in 0.02% Azide/PBS at 4° C.

Histology

Tissues were cryoprotected in 30% sucrose/PBS overnight at 4° C. After embedment in OCT the following day, 20 μm sections were directly mounted onto slides. For X-gal staining, tissues were rinsed in PBS and Triton-X/PBS and stained in 0.1% X-gal solution overnight at 30-35° C. After staining sections were rinsed and counterstained with neutral red, dehydrated and mounted with coverslips. For co-labeling of X-gal with markers using immunohistochemistry, standard IHC procedure was followed and the X-gal stain was performed either prior to primary antibody incubation or between primary and secondary antibodies, depending on the strength of the X-gal stain. X-gal stains blue any cells that have recombined the Gt(ROSA26)^{tm1Sor} locus due to iCre recombinase activity and thus expressing the ß-galactosidase protein.

Knock-in Mouse Generation and Analysis

Expression Vector

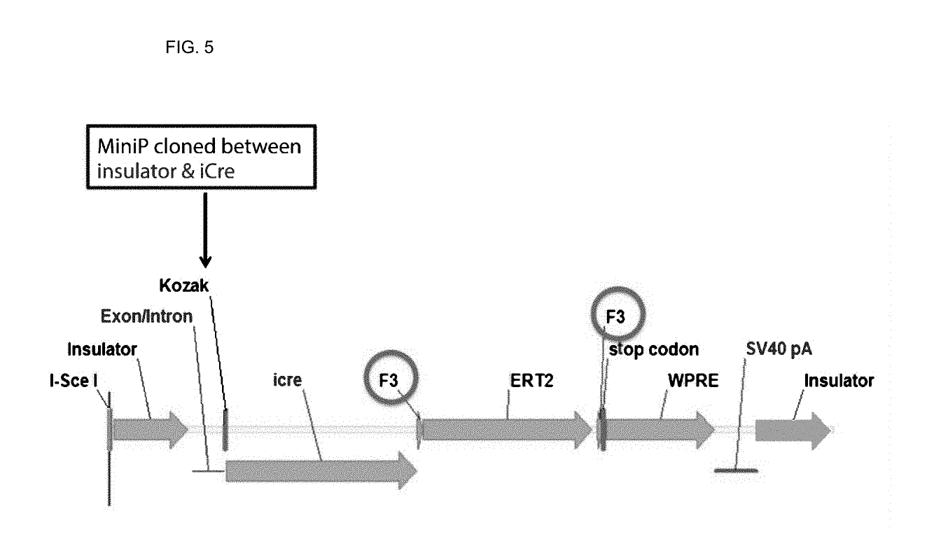

The nucleic acid fragment corresponding to (SEQ ID NO: 1) was inserted into the multiple cloning site of a targeting vector (driving the inducible CreER^{T2} reporter, see FIG. 5) to produce the expression vector (containing Ple264) used in the experiments.

Derivation of mEMS4855.02 Embryonic Stem Cells

Blastocysts were obtained from natural mating of B6/NTac-Aw-J, Hprt^{b-m3} homozygous females to C57BL/6NTac males at 3.5 dpc. Blastocysts were flushed from uterine horns as per (Hogan, Beddington et al. 1994), cultured in EmbryoMax® KSOM with 1/2 Amino Acids, Glucose and Phenol Red (Cat # MR-121, Millipore/Chermicon, Temecula, Calif.) for 3-5 h, and then transferred onto mitomycin C (mitC; Cat#M4287, Sigma, Oakville, ON) mitotically inactivated B6-Hprt^{b-m3}, B6129F1, or 129 mouse embryonic feeders (MEFs) derived from 13.5-day post-coital embryos (Ponchio, Duma et al. 2000) in 96-well plates containing KSR-ESC (Knockout™ D-MEM, Cat#10829-018, Invitrogen, Burlington, ON) with 2 mM L-glutamine (Cat#25030-081, Invitrogen, Burlington, ON), 0.1 mM MEM nonessential amino acid solution (Cat#11140-050, Invitrogen, Burlington, ON) and 16% Knockout™ Serum Replacement (Cat#10828-028, Invitrogen, Burlington, ON)) media (MEF media was replaced 3-5 hour prior to transfer). Blastocysts were cultured as per (Cheng, Dutra et al. 2004) with the following modifications: Cells were cultured for 7-9 days in KSR-ESC with minimal disturbance (checked on day 2 to determine if the blastocysts had ‘hatched’ out of the zona pellucida) and no media changes. Blastocysts which hatched and had a well-developed ICM (inner cell mass) were treated with 20 μl 0.25% trypsin-EDTA (Invitrogen, Burlington, ON) for 5 min at 37° C., triturated with a 200 μl Pipetman, inactivated with 30 μl 0.5 mg/ml soybean trypsin inhibitor (Invitrogen, Burlington, ON), and brought up to 200 μl with KSR-ESC, then transferred individually to a 24-well MEF plate containing 1800 μl KSR-ESC, for a total volume of 2 ml. Beginning 4 days later, KSR-ESC media was replaced with FBS-ESC media (DMEM (Cat #11960-069, Invitrogen, Burlington, ON) with 2 mM L-glutamine (Invitrogen, Burlington, ON), 0.1 mM MEM nonessential amino acid solution (Invitrogen, Burlington, ON), 16% ES Cell Qualified fetal bovine serum (FBS, Invitrogen, Burlington, ON), 1000 U ESGRO-LIF (Millipore, ESG1107) and 0.01% β-mercaptoethanol (Sigma, Oakville, ON)) in 25%, 50%, 75% proportions (respectively) to adapt the cells to FBS-containing media. On day 7 the cells were trypsinized to one well of a 24 well plate containing 1 ml of 100% FBS-ESC media, with daily media replacement. Once confluent, wells containing ESC colonies were expanded 3×24 wells (with MEFs), then passaged to 3×24 (with MEFs) and 3×12 well (plastic—no MEFs) for DNA analysis. Once confluent, the 3×24 wells were combined, aliquoted (3 vials), and frozen in ESC-freeze media (50% FBS, 40% FBS-ESC media, 10% DMSO (Sigma, Oakville, ON), and the 3×12 well treated with lysis buffer (Fisher Scientific, Ottawa, ON), mixed and aliquoted. Cultures were genotyped for X & Y chromosomes (Clapcote and Roder 2005) and Hprtb-m3 and WT alleles. B6/NTac-Aw-J/Aw-J, Hprt<b-m3>/Y ESC cell lines (mEMS4855.02) were identified.

Knock-in at the Hprt Locus

The expression vector plasmid DNA was purified with Qiagen Maxi Kit (Qiagen, Mississauga, ON), resuspended in 10:1 Tris-EDTA (TE, pH7.0) buffer, and linearized with I-Scel (New England Biolabs, Pickering, ON). Linearized plasmid DNA was resuspended in 85 μl of TE 10:0.1) to a final concentration of 187.5 ng/μl. Ple(264) was targeted in our in-house derived mEMS4855.02 cell line. ESCs were grown to confluence on 4-6 T75 flasks of mitC treated Hprt^{b-m3} mouse embryonic feeders (MEFs) in FBS-ESC media. ESCs (1.7-2.5×107) in 720 μl 1×PBS were added to the linearized DNA and electroporated in a 4 mm electroporation cuvette (Bio-Rad Genepulser, Mississauga, ON), at 240 V, 50 μF, 6-10 msec pulse, immediately resuspended in a total volume of 5 ml of FBS-ESC media and plated onto 5×100 mm dishes of mitC B6129F1 MEFs in a total volume of 12 ml per 100 mm dish. 24-36 h post-electroporation, correctly targeted homologous recombinants were selected for using HAT media (FBS-ESC media containing 1×HAT ((0.1 mM sodium hypoxanthine, 0.4 mM aminopterin, 0.16 mM thymidine), Cat#21060-017, Invitrogen, Burlington, ON). HAT media was changed every day for the first 3 days, and then every 3rd day thereafter, for up to 10 days. Individual colonies were counted and, typically, no more than 2 isolated colonies were picked per 100 mm dish to optimize for independent homologous recombination events. These colonies were expanded under standard protocols for verification of the desired recombination event.

Derivation of Knock-in Mice

Chimeric mice from targeted ESCs were generated by microinjection (Hogan, Beddington et al. 1994) into E3.5 blastocysts followed by implantation into the uterine horns of 2.5 day pseudopregnant ICR females. Chimeras were identified and coat color chimerism determined as outlined below.

Male chimeras derived from the ESC cell lines were mated with C57BL/6J females, and germline transmission was identified in the former case by the transmission of the dominant Aw (white bellied agouti) allele, making the progeny appear brown with a cream belly. Non-germline progeny from the cross to B6 were homozygous for the recessive a (nonagouti) allele and appeared black. All germline female offspring carry the knock-in X Chromosome and were mated with B6 males. N2 offspring were analyzed for the presence of the KI allele by PCR. Germline mice were crossed with B6-CAG-Flpo mice (C57BL/6-Tg(CAG-Flpo)1Afst/Mmcd; MMRRC Stock#032247-UCD) to remove the ER^{T2} inducibility cassette. These mice were then crossed to N1 or N2 tdTomato reporter mice (B6.Cg-Gt(ROSA)26Sortm14(CAG-tdTomato)Hze/J; JAX Stock#007914), genotyped, and used for expression analysis.

Reporter Gene Detection

Adult mice with hemi- or heterozygous MiniPromoter genotype, and with the tdTomato reporter (het), were perfused alongside age-matched control mice using 4% paraformaldehyde (PFA) as previously described (Young, Berry et al. 2002). Whole brains and eyes were dissected out and post-perfusion immersion fixed with PFA for 2 hours at 4° C.

Brain and eyes were removed from the solution and blotted with a KimWpe before embedment in Optimal Cutting Temperature (OCT) alongside positive and negative controls. 12 μm sections were taken using a Microm HM 550 cryostat and directly mounted onto SuperFrost Plus microscope slides. Slides were counterstained with the nuclear stain DAPI and mounted with coverslips. Fluorescent image acquisition and analysis was done using either an Olympus BX61 microscope or a Leica SP5 II laser confocal microscope.

### Example 1—Selection of NR2E1 Mini-Promoter Elements

The expression vector comprising the Ple264 mini-promoter contained three different regulatory elements from the human NR2E1 upstream regulatory region (SEQ ID NOs: 3, 4 and 5). These three regulatory regions were fused to a basal promoter (SEQ ID NO: 2) that spans the translational start site of exon 1 of the human NR2E1 gene. The expression vector comprising the Ple140 mini-promoter contained an additional two regulatory elements.

### Example 2—Expression of Reporter in Brain and Eye by Ple264 Mini-Promoter

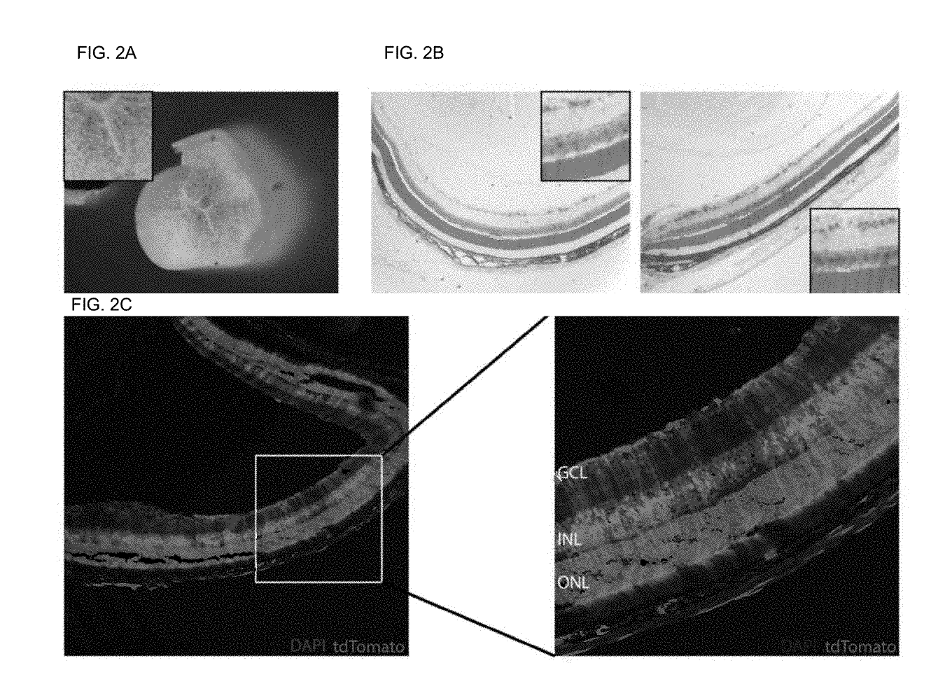

The vEMS23 ssAAV9 virus was injected intravenously into P0 mice containing the Gt(ROSA26)Sor^{tm1Sor} locus. This virus contains the Ple264 Mini-Promoter driving expression of the iCre recombinase. Upon expression, recombination of the ROSA26 locus via iCre results in expression of β-galactosidase (lacZ). Eyes were harvested at P21, stained for lacZ activity using the X-gal substrate (blue product) and counter-stained with neutral red to visualize cell nuclei. Expression was observed in the ganglion cell layer, but predominantly in cell bodies of the inner nuclear layer with long processes extending the span of the neural retina consistent with, and unique to, Müller glial cells (FIG. 2A,B). We further tested this MiniPromoter, Ple264, using a knock-in strategy at the Hprt locus whereby the Ple264 promoter drives expression of iCre recombinase and recombines the Ai14 allele at the ROSA26 locus (JAX Stock#007908. Full m14(CAG-tdTomato)Hze strain name: B6; 129S6-Gt(ROSA)26Sor^{tm14(CAG-tdTomato)Hze}/J); this results in the expression of the reporter tdTomato from a ubiquitous CAG promoter in cells that have undergone recombination. This system also demonstrated Müller glia cell expression in the adult retina (FIG. 2C).

### Example 3—Expression of Reporter in Brain by Ple140 Mini-Promoter

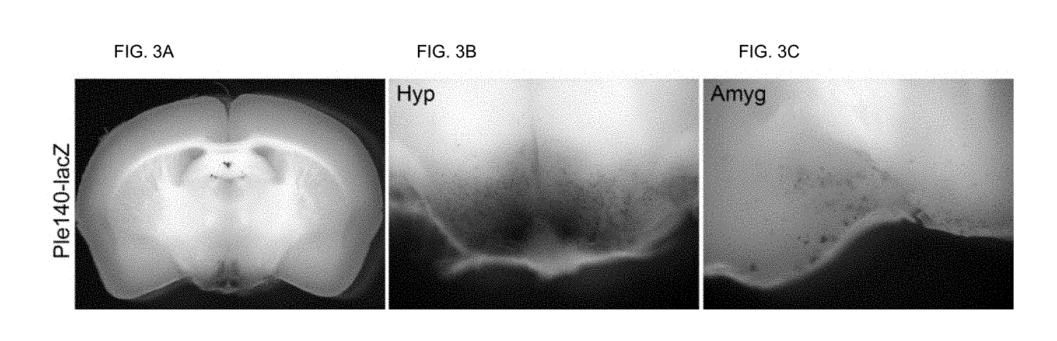

The Ple140 mini-promoter (SEQ ID NO: 6) is similar to the Ple264 mini-promoter, except that it has 2 additional upstream regulatory elements added to it. The 5 upstream regulatory elements are fused to each other in their natural orientation and sequential arrangement as found in the endogenous promoter sequence, but with all intervening sequences removed. The expression vector comprising the Ple140 mini-promoter was introduced into mouse embryonic stem cells (ESCs) at the Hprt locus as described earlier in Portales-Casamar et al. PNAS (2010) 170(38): 16589-94. The ESCs were used to generate genetically modified mice containing Ple140 NR2E1 mini-promoters driving reporter gene expression. Immunohistochemical and immunofluorescence analysis of mouse brain tissue slices revealed lacZ reporter expression in the brain. (FIG. 3). Immunohistochemical and immunofluorescence analysis of mouse eye tissue revealed no detectable lacZ reporter expression. Thus, the inclusion of additional regulatory elements into Ple140, as compared to the smaller Ple264, appears to eliminate expression in the eye. This further illustrates the novelty and utility of Ple264, particulary for driving expression in the eye.

### REFERENCES

Abrahams B S, Mak G M, Berry M L, Palmquist D L, Saionz J R, Tay A, Tan Y H, Brenner S, Simpson E M, Venkatesh B. 2002. Novel vertebrate genes and putative regulatory elements identified at kidney disease and NR2E1/fierce loci. Genomics 80: 45-53.
Miyawaki T, Uemura A, Dezawa M, Yu R T, Ide C, Nishikawa S, Honda Y, Tanabe Y, Tanabe T. 2004. Tlx, an orphan nuclear receptor, regulates cell numbers and astrocyte development in the developing retina. Journal of Neuroscience 24: 8124-8134.
Monaghan A P A, Grau E E, Bock D D, Schütz G G. 1995. The mouse homolog of the orphan nuclear receptor tailless is expressed in the developing forebrain. Development 121: 839-853.
Schmouth J-F, Bonaguro R J, Corso-Diaz X, Simpson E M. 2012. Modelling human regulatory variation in mouse: finding the function in genome-wide association studies and whole-genome sequencing. eds. J.-F. Schmouth, R. J. Bonaguro, X. Corso-Diaz, and E. M. Simpson. PLoS Genet 8: e1002544.
Shi Y, Chichung Lie D, Taupin P, Nakashima K, Ray J, Yu R T, Gage F H, Evans R M. 2004.
Expression and function of orphan nuclear receptor TLX in adult neural stem cells. Nature 427: 78-83.
Soriano P. 1999. Generalized lacZ expression with the ROSA26 Cre reporter strain. Nat Genet 21: 70-71.

1-4. (canceled)

5. An isolated polynucleotide comprising a NR2E1 mini-promoter with at least 95% sequence identity to SEQ ID NO: 1.

6. The isolated polynucleotide of claim 5, operably linked to an expressible sequence.

7. A vector comprising the isolated polynucleotide of claim 5.

8. A cell comprising the vector of claim 7.

9. The cell of claim 8, wherein the vector is stably integrated into the genome of the cell.

10. The cell of claim 8, wherein the cell is a stem cell, a retinal cell or a brain cell.

11. A method of expressing a sequence of interest, the method comprising operably linking the sequence of interest to the polynucleotide of claim 5; and introducing into a cell permissive for expression from the NR2E1 mini-promoter.

12. A method of treatment of a subject having a disease or condition of the nervous system, in particular the eye, the method comprising administering to the subject a therapeutically effective dose of a composition comprising a polynucleotide of claim 5.

13. A method of treatment of a subject having a disease or condition of the nervous system, in particular the eye, the method comprising administering to the subject a therapeutically effective dose of a composition comprising a cell of claim 8.
